(19) United States
(12) Patent Application Publication
(10) Pub. No.: US 2005/0069499 A1
(43) Pub. Date: Mar. 31, 2005

(54) Foamable compositions, processes of preparing same and uses thereof

(76) Inventors: Moshe Arkin, Kfar Shmaryahu (IL); Rina Uzan, Beer Sheva (IL); Natalie Grabarnick, Beer Sheva (IL); Benjamin Schneider, Rehovot (IL); Stephen Cherkez, Caesarea (IL); Eilon Asculai, Lehavim (IL); Amira Zeevi, Omer (IL); Chalil Abu-Gnim, Beer Sheva (IL)

Correspondence Address: G.E. EHRLICH (1995) LTD.;c/o ANTHONY CASTORINA, SUITE 207, 2001 JEFFERSON DAVIS HIGHWAY, ARLINGTON, VA, 22202, US

(21) Appl. No.: 10/812,356

(22) Filed: Mar. 30, 2004

Related U.S. Application Data
(60) Provisional application No. 60/505,426, filed on Sep. 25, 2003.
(60) Provisional application No. 60/527,278, filed on Dec. 8, 2003.

(51) Int. Cl. A61L 9/04; A61K 31/573

(52) U.S. Cl. 424/45; 514/171

(57) ABSTRACT

A foamable composition being devoid of CFCs and buffer, which produces quick-breaking foam and optionally comprises pharmaceutically active ingredients such as corticosteroids is disclosed. Methods of treatment using the same and processes of manufacturing the same are also disclosed.

This application claims the benefit of U.S. Provisional Patent Application No. 60/505,426 filed Sep. 25, 2003 and U.S. Provisional Patent Application No. 60/527,278 filed Dec. 8, 2003 both which are hereby incorporated by reference as if fully set-forth herein.

### FIELD AND BACKGROUND OF THE INVENTION

The present invention relates to the field of pharmacology and more particularly, to a foamable composition especially useful for topical delivery of medicaments such as corticosteroids, methods for the preparation thereof and methods of treatment using the same.

The delivery of certain medicaments topically is well known in the art. Topical forms of medicament delivery include lotions, creams, pastes, gels, ointments, salves, milks, tinctures and solutions. The convenience of use and efficacy of a topical composition are in a large part determined by the form of the composition.

The challenge in topically applying a composition is achieving percutaneous penetration of the active agent to the site of treatment, in many cases the epidermis. At the same time, it is important that a composition should have desirable characteristics such as ease of application, smooth texture and application should result in no irritation, discomfort or inconvenience. Desirably, the composition should not leave a residue where applied.

Topical compositions in forms such as gels, ointments, lotions, creams, salves and pastes are often very viscous, requiring substantial rubbing to achieve penetration of the active agent to the affected skin layer, an act which often results in discomfort and irritation. Non-viscous creams and lotions require quick and dexterous application as they are inclined to flow off the site of treatment before penetration of the active ingredient is achieved.

In contrast, foams are well suited for the topical application of compositions. The advantages of foamable compositions for the topical application of pharmaceutical are well known in the art. In the field of pharmacology the rigid yet fluid nature of foamable compositions is desirable, allowing a foam to be applied in any orientation without run-off as well as the allowing the convenient application of foam evenly over large surfaces. Lastly, when a foam is applied, even by rubbing, onto damaged or sensitive skin, the foam acts as a cushion, allowing spreading without direct physical contact.

Foamable compositions are generally single or multi-phase liquids provided in a container, often together with a propellant to transport the composition from the container, transforming it into foam upon application. Another technique for the application of foams includes the “bag-in-a-can”. In such products, the product may contain a low-boiling hydrocarbon like isopropane that has a boiling point of about 28° C. Application and agitation of the product at body temperature cause the isopropane to vaporize and generate the foam similar to a pressurized aerosol foaming system.

The physical characteristics of foam formed by a foamable composition are dependent upon the nature and relative amounts of components such as solvents, propellants and surfactants. One of the most important characteristics is whether a foam is long-lasting or quick-breaking, a qualitative description of the behavior of the foam towards shearing action encountered, for example, when the foam is rubbed into or spread over a surface onto which it has been applied, such as skin.

One method of producing quick-breaking foams is by the use of a foamable composition with a relatively high alcohol content. Upon contact with the skin the alcohol in such foams evaporates. Thus the foam relatively quickly collapses into a liquid when disturbed (e.g. by rubbing) or when warmed by body heat driving the active agent through the skin layers to the site of treatment. This allows a user to quickly dispense a desired amount to achieve a quick effect.

Corticosteroids are well known anti-inflammatory compounds, which are recognizably utilized in the treatment of acute inflammatory diseases such as allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria (insect bite reactions), palmoplantar psoriasis, discoid lupus erythematosus, chronic hypertrophic lichen planus, granuloma annulare and keloid scars.

The topical application of corticosteroid compositions for the treatment of these and other skin ailments is well established in the art and is effected, inter alia, using foamable compositions.

In one example, Woodford et al. J. Pharmaceutical Science 66: 99-103 (1977) describe a corticosteroid foamable composition containing betamethasone benzoate, betamethasone valerate, clobetasol propionate, desonide, triamcinolone acetonide, flumethasone pivalate and hydrocortisone-17-butyrate, which produces a quick-breaking foam using CFC propellants.

U.S. Pat. No. 3,856,956 also teaches a corticosteroid foamable composition that includes CFC propellants. However, as it is well known that CFC gases damage the environment, the above foamable corticosteroid compositions were considered highly disadvantageous and efforts have been made to produce compositions devoid of CFC gases.

U.S. Pat. No. 5,352,437 teaches a crackly foamable composition, using n-butane as a propellant instead of CFC. The composition includes between 0.05% and 10% of a low hydrocarbon alcohol or a glycol and a high content of the propellant (between 60 and 95 weight percentages), and may optionally further include an active ingredient.

In the art it is known that corticosteroids (as well as other pharmaceutically active compounds, especially esters) tend to decompose or isomerize to less than ideal structures, (see, for example, U.S. Pat. No. 5,914,122). It is thus important when providing a pharmaceutical composition, to evaluate the stability of the pharmaceutically active compound used therein.

U.S. Pat. No. 6,126,920 teaches a corticosteroid foamable composition that comprises an aliphatic alcohol (40%-90% w/w), water (10%-40% w/w), a fatty alcohol (0.5%-10% w/w), a surface-active agent (0.1%-55% w/w) and a buffer where an included corticosteroid is stable.

In U.S. Pat. No. 6,126,920 the nature of the propellant is not discussed but two commercial products based on this patent, Luxiq® and Olux® (Connetics®, Palo Alto, Calif., USA), use a butane/propane mixture as a propellant and have no CFC based propellant.

In U.S. Pat. No. 6,126,920 it is reported that the only way to ensure the stability of the most active isomer of the active ingredient of the composition is by including a buffering agent selected from amongst acetic acid/sodium acetate, citric acid/sodium citrate and phosphoric acid/sodium phosphate, and it is desirable generally to buffer the composition to a pH of 3.0-6.0, preferably 4.0-5.0 and to this end the buffering agent may preferably be present in an amount of 0.01-1.0% w/w, more preferably 0.05-0.2% w/w. For betamethasone valerate it is reported that particularly preferred is an anhydrous citric acid/potassium citrate, to buffer the composition to pH 4.5, so as to stabilize the more active 17-valerate ester over the less active 21-valerate ester.

The use of a buffer system obviously increases the complexity and costs of manufacture of a composition made according to the teachings of U.S. Pat. No. 6,126,920.

There is thus a widely recognized need for, and it would be highly advantageous to have, a foamable composition that can be used to topically deliver pharmaceutical, which does not use CFC propellants and is further devoid of the disadvantages of compositions known in the art.

### SUMMARY OF THE INVENTION

The present invention successfully addresses the above-recited needs by providing an innovative foamable composition that is devoid of a CFC propellant as well as a buffer agent.

According to the present invention there is provided a foamable pharmaceutical composition comprising at least one corticosteroid active ingredient, a non-CFC propellant and an acceptable carrier configured to generate a quick-break foam, the composition being devoid of a buffering agent.

According to another aspect of the present invention, the carrier comprises at least one hydrocarbon alcohol, at least one fatty alcohol, at least one surface-active agent and water.

According to a feature of the present invention the at least one hydrocarbon alcohol has from one to ten, preferably from one to six, carbon atoms.

According to a feature of the present invention the at least one hydrocarbon alcohol is an aliphatic hydrocarbon alcohol, preferably selected from the group consisting of methanol, ethanol, n-propanol, isopropanol, n-butanol, sec-butanol, isobutanol and t-butanol and mixtures thereof. The concentration of the at least one hydrocarbon alcohol preferably ranges between about 40 weight percentages and about 90 weight percentages of the total weight of the composition. More preferably it is between about 50 weight percentages and about 70 weight percentages of the total weight of the composition.

According to a feature of the present invention the at least one fatty alcohol has between 10 and 22 carbon atoms, and is preferably selected from the group consisting of cetyl alcohol, stearyl alcohol, lauryl alcohol, myristyl alcohol, palmityl alcohol and mixtures thereof. According to a feature of the present invention the concentration of the at least one fatty alcohol ranges between about 0.1 weight percentage and about 20 weight percentages of the total weight of the composition. Preferably it is between about 0.5 weight percentage and about 10 weight percentage of the total weight of the composition.

According to a feature of the present invention the concentration of the water ranges between about 10 weight percentages and about 40 weight percentages of the total weight of the composition.

According to a feature of the present invention the at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof. According to a feature of the present invention the concentration of the at least one surface-active agent ranges between about 0.1 weight percentage and about 60 weight percentages of the total weight of the composition. Preferably it is between about 0.2 weight percentage and about 15 weight percentages of the total weight of the composition.

According to a feature of the present invention the concentration of the non-CFC propellant ranges between about 1 weight percentage and about 40, preferably 20, weight percentages of the total weight of the composition.

According to a feature of the present invention the non-CFC propellant is selected from a group of propellants consisting of nitrous oxide, carbon dioxide, nitrogen, propane, iso-butane, n-butane, isopentane, n-pentane, dimethyl ether and any combination thereof. Preferably, the non-CFC propellant comprises a mixture of propane, n-butane and isobutane.

According to a feature of the present invention, the foamable pharmaceutical composition of the present invention has a pH of between about 4.0 and about 7.0.

According to another feature of the present invention, the foamable pharmaceutical composition further comprises at least one humectant such as, but not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

According to yet another feature of the present invention, the foamable pharmaceutical composition further comprises at least one pH-adjusting agent such as, but not limited to, adipic acid, glycine, calcium hydroxide, magnesium aluminometasilicates, hydrochloric acid, and any combination thereof. Preferably, the at least one pH adjusting agent is selected from the group of acids consisting of citric acid, phosphoric acid, lactic acid, sorbic acid and tartaric acid, whereby the acid being the only source of a respective anion in the composition.

According to a feature of the foaniable pharmaceutical composition of the present invention, the at least one corticosteroid active ingredient is selected from the group consisting of alclometasone dipropionate, amcinonide, beclomethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone, fluprednidene acetate, flurandrenolone, fluticasone, halcinonide, hydrocortisone, hydrocortisone acetate, hydrocortisone butyrate, methylprednisolone acetate, mometasone furoate, triamcinolone acetonide, and mixtures thereof., preferably selected from the group consisting of betamethasone valerate and clobetasol propionate. The concentration of the at least one corticosteroid active ingredient preferably ranges between about 0.01 weight percentage and about 1 weight percentage of the total weight of the composition. More preferably, it ranges between about 0.05 weight percentage and about 0.2 weight percentage of the total weight of the composition.

According to still another feature of the present invention the foamable pharmaceutical composition of the present invention is packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. The condition is preferably selected from the group consisting of acute inflammatory diseases, allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

According to an embodiment of the foamable pharmaceutical composition of the present invention, the carrier comprises ethanol, cetyl alcohol, stearyl alcohol, polysorbate 60 and water; the non-CFC propellant comprises a mixture of propane, n-butane and isobutane, at a concentration that ranges between about 1 weight percentage and about 40 weight percentages of the total weight of the composition; and the at least one corticosteroid active ingredient is clobetasol propionate or betamethasone valerate, at a concentration that ranges between about 0.01 (preferably 0.05) weight percentage and about 1 (preferably 0.2) weight percentage of the total weight of the composition.

According to one preferred embodiment of the foamable pharmaceutical composition present invention the concentration of the ethanol ranges between about 40 weight percentages and about 90 weight percentages of the composition, the concentration of the cetyl alcohol ranges between about 0.1 and about 20 weight percentages of the composition, the concentration of the stearyl alcohol ranges between about 0.1 and about 20 weight percentages of the composition, the concentration of the polysorbate 60 ranges between about 0.1 and about 60 weight percentages of the composition and the concentration of the water ranges between about 10 and about 40 weight percentages of the composition.

According to another preferred embodiment of the foamable pharmaceutical composition of the present invention the concentration of the ethanol ranges between about 50 weight percentages and about 70 weight percentages of the composition, the concentration of the cetyl alcohol ranges between about 0.5 and about 10 weight percentages of the composition, the concentration of the stearyl alcohol ranges between about 0.1 and about 10 weight percentages of the composition, the concentration of the polysorbate 60 ranges between about 0.2 and about 15 weight percentages of the composition and the concentration of the water ranges between about 10 and about 40 weight percentages of the composition.

According to still another preferred embodiment of the foamable pharmaceutical composition of the present invention the concentration of the ethanol ranges between about 56 weight percentages and about 65 weight percentages of the composition, the concentration of the cetyl alcohol ranges between about 0.9 and about 1.3 weight percentages of the composition, the concentration of the stearyl alcohol ranges between about 0.4 and about 0.6 weight percentage of the composition, the concentration of the polysorbate 60 ranges between about 0.2 and about 0.6 weight percentage of the composition and the concentration of the water ranges between about 31 and about 36 weight percentages of the composition, propylene glycol in a concentration of between about 1 and about 3 weight percentages of the composition, propane/butane/isobutene as a non-CFC propellant in a concentration of between about 4 and about 5 weight percentages of the composition, clobetasol propionate in a concentration between about 0.01 and about 0.1 weight percentage of the composition, whereby the composition has a pH of between about 6.5.

According to still another preferred embodiment of the foamable pharmaceutical composition of the present invention the concentration of the ethanol ranges between about 56 weight percentages and about 65 weight percentages of the composition, the concentration of the cetyl alcohol ranges between about 0.9 and about 1.3 weight percentages of the composition, the concentration of the stearyl alcohol ranges between about 0.4 and about 0.6 weight percentage of the composition, the concentration of the polysorbate 60 ranges between about 0.2 and about 0.6 weight percentage of the composition and the concentration of the water ranges between about 31 and about 36 weight percentages of the composition, propylene glycol in a concentration of between about 1 and about 3 weight percentages of the composition, propane/butane/isobutene as a non-CFC propellant in a concentration of between about 4 and about 5 weight percentages of the composition, clobetasol propionate in a concentration of between about 0.01 and about 0.1 weight percentage of the composition and lactic acid, whereby the concentration of the lactic acid is sufficient for adjusting the pH of the composition to between about 5.9 and about 6.1.

Although the carrier of the present invention is exceptionally useful in implementing the innovative foamable pharmaceutical composition of the present invention for application of a corticosteroid active ingredient, the carrier of the present invention is also useful in implementing a general foamable composition, with or without an active ingredient.

Hence, According to another aspect of the present invention there is provided a foamable composition comprising the non-CFC propellant and the carrier described hereinabove, the composition being devoid of a buffering agent.

According to an embodiment of this aspect of the present invention the foamable composition further comprises at least one pharmaceutically active ingredient, which is preferably a pH sensitive pharmaceutical active ingredient such as, but not limited to corticosteroids, non-steroidal anti-inflammatory drugs, and the like.

The foamable composition of this aspect of the present invention can be packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition.

According to still another aspect of the present invention there is provided a method of treatment comprising administering (preferably topically or rectally) a therapeutically effective amount of the foamable pharmaceutical composition or the foamable composition as described hereinabove to a mammal, especially a human, in need thereof.

According to one aspect of the method present invention the administering is effected by passing the composition from a first volume having a first pressure (e.g. a pressurized container) through a passage (e.g. a valve) into a volume having a second pressure, the second pressure being lower than the first pressure (e.g. the outside environment) so as to effect foaming of the composition. Preferably the foamed composition is administered onto a surface, such as skin.

According to an embodiment of the method of the present invention, the need for which the composition of the present invention is administered arises from a medical condition, the need selected from the group of needs consisting of curing the condition, treating the condition, preventing the condition, treating symptoms of the condition, curing symptoms of the condition, ameliorating symptoms of the condition, treating effects of the condition, ameliorating effects of the condition, and preventing results of the condition.

According to one embodiment of the method of the present invention the need for which the composition is administered arises from a medical condition selected from the group consisting of acute inflammatory diseases, allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

According to a feature of the method of the present invention, a preferred active ingredient is a corticosteroid active ingredient selected from the group consisting of alclometasone dipropionate, amcinonide, beclomethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone, fluprednidene acetate, flurandrenolone, fluticasone, halcinonide, hydrocortisone, hydrocortisone acetate, hydrocortisone butyrate, methylprednisolone acetate, mometasone furoate, triamcinolone acetonide, and mixtures thereof. According to a preferred embodiment the preferred active corticosteroid is selected from the group consisting of betamethasone valerate and clobetasol propionate.

According to a feature of the method of the present invention the concentration of the at least one corticosteroid active ingredient ranges between about 0.01 (preferably 0.05) weight percentage and about 1 (preferably 0.2) weight percentage of the total weight of the composition.

According to yet another aspect of the present invention there is provided a process of preparing a foamable composition or a foamable pharmaceutical composition as described above, which comprises obtaining a carrier by mixing at least one hydrocarbon alcohol, at least one fatty alcohol, at least one surface active agent, and water; placing the carrier in a pressure-resistant vessel; placing an amount of at least one non-CFC propellant into the pressure-resistant vessel; and sealing the pressure-resistant vessel.

According to one embodiment of the process of the present invention, the process further comprises, prior to the placing of the carrier in the vessel, admixing with the carrier an appropriate corticosteroid (or other) active ingredient.

According to another embodiment of the process of the present invention, the process further comprising, prior to the placing of the carrier in the vessel, admixing with the carrier at least one humectant, as is described hereinabove According to yet another embodiment of the process of the present invention, obtaining a carrier includes heating a mixture of the at least one hydrocarbon alcohol, the at least one fatty alcohol, the at least one surface-active agent and the water, at a temperature of at least 30° C., more preferably at least 40° C.

According to still another embodiment of the process of the present invention, obtaining a carrier comprises: mixing the water, the at least one fatty alcohol and the at least one surface-active agent, so as to obtain a clear aqueous solution; and adding the at least one hydrocarbon alcohol to the aqueous solution, to thereby obtain the carrier. According to a feature of this embodiment, the mixing further includes heating the aqueous solution at a temperature of at least about 40° C., preferably at least about 60° C. According to a further feature of this embodiment, adding the hydrocarbon alcohol is preferably performed while heating the aqueous solution at a temperature of at least about 30° C., more preferably at a temperature of at least about 39° C.

According to an additional embodiment of the process of the present invention, obtaining a carrier comprises: mixing the hydrocarbon alcohol, the at least one fatty alcohol and the at least one surface-active agent, so as to obtain a clear alcoholic solution; and adding the water to the alcoholic solution, to thereby obtain the carrier. According to a feature of the present invention, adding the water is performed while heating the alcoholic solution at a temperature of at least about 30° C., preferably at a temperature of at least about 40° C.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

With specific reference now to the drawings in detail, it is stressed that the particulars shown are by way of example and for the purposes of illustrative discussion of the preferred embodiment of the present invention only, and are presented in the cause of providing what is believed to be the most useful and readily understood description of the principles and conceptual aspects of the invention. In this regard, no attempt is made to show structural details of the invention in more detail that is necessary for a fundamental understanding of the invention, the description taken with the drawings making apparent to those skilled in the art how the several forms of the invention may be embodied in practice.

In the drawings:

FIG. 1 is a comparison of titration of a composition of the present invention (a), a composition without a pH-adjusting agent (b) and a prior-art buffered composition (c); and

FIG. 2 is a comparison of the back titration of a composition of the present invention (a), a composition without pH-adjusting agent (b) and a prior-art buffered composition (d).

### DESCRIPTION OF THE PREFERRED EMBODIMENTS

The present invention is of a foamable composition that is useful, amongst other uses, as a carrier for topical pharmaceutical compositions, especially those containing corticosteroids. The present invention includes the preparation of the composition of the present invention. The present invention also includes methods of treatments using a foamable composition of the present invention.

The principles, uses and implementations of the present invention are better understood with reference to the accompanying descriptions and examples.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

As used herein, the term “comprising” means that other steps and ingredients, which do not affect the final result, can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As is discussed in detail hereinabove, quick-breaking foams present an advantageous form for topical dispensation of pharmaceutically active ingredients, by providing quick and accurate dosage, high penetration, convenient application to large areas of the body surface, ease of application, economy in use, and general suitability for both smooth and hirsute skin. As is further discussed hereinabove, the foamable compositions known in the art for topically applying active ingredients such as corticosteroids typically either comprise hazardous CFC propellants, or, when comprising non-CFC propellants, typically include a buffering agent, which is aimed at stabilizing the active ingredient within the composition.

The present inventors have now surprisingly found that foamable compositions of corticosteroids, which are designed to provide a quick break foam using non-CFC propellants, can be formulated without a buffering agent, while still maintaining the stability of the active ingredients.

Hence, unlike prior-art compositions, the foamable compositions of the present invention are exceptionally suitable for use in dispensing pharmaceutically active agents, especially such ingredients that are pH sensitive. This arises from the fact that, for a composition of the present invention, a desired pH is achieved and maintained without the addition of a buffering agent. When a foamable composition of the present invention includes a pharmaceutically active agent, the composition is referred to herein as a foamable pharmaceutical composition.

Thus, according to one aspect of the present invention, there is provided a foamable pharmaceutical composition, which comprises one or more corticosteroid active ingredient(s), a non-CFC propellant and an acceptable carrier configured to generate a quick-break foam, and which is devoid of a buffering agent.

As used herein, the phrase “acceptable carrier” describes a carrier that does not cause significant irritation to an organism and does not abrogate the biological activity and properties of the applied active ingredient.

As used herein, the phrase “quick-break foam” describes a foam that collapses when exposed to shearing action as is sustained when the foam is rubbed into or spread over a body surface onto which it has been dispensed.

In a preferred embodiment of the present invention, the carrier comprises at least one hydrocarbon alcohol, at least one fatty alcohol, at least one surface-active agent and water.

As used herein, the phrase “hydrocarbon alcohol” describes a hydrocarbon that is substituted by one or more hydroxyl groups. The hydrocarbon is preferably aliphatic, i.e., an alkyl, having between 1 and 10 carbon atoms, preferably between 1 and 6 carbon atoms, thus being a low hydrocarbon alcohol. The alkyl can be branched or un-branched, saturated or unsaturated, preferably saturated.

Representative examples of hydrocarbon alcohols that are usable in the context of the present invention include, without limitation, methanol, ethanol, n-propanol, isopropanol, n-butanol, sec-butanol, isobutanol and t-butanol and any combination thereof, with ethanol being preferred.

The concentration of the hydrocarbon alcohol preferably ranges between about 40 weight percentages and about 90 weight percentages of the total weight of the composition, more preferably between about 40 weight percentages and about 70 weight percentages, more preferably between about 50 weight percentages and about 70 weight percentages, more preferably between about 55 weight percentages and about 65 weight percentages, more preferably between about 58 weight percentages and about 62 weight percentages and most preferably it is about 60 weight percentages of the total weight of the composition.

As used herein throughout, the phrase “weight percentage(s)” describes the weight percentage(s) (of an ingredient) of the total weight of a composition containing the same.

As used herein the term “about” refers to ±10%.

As used herein, the phrase “fatty alcohol” describes a non-aromatic hydrocarbon alcohol having at least ten carbon atoms and no more than one alcohol group.

In general a fatty alcohol used in implementing a composition of the present invention has between 10 and 22 carbon atoms. Representative examples of fatty alcohols that are usable in the context of the present invention include, without limitation, cetyl alcohol, stearyl alcohol, lauryl alcohol, myristyl alcohol, palmityl alcohol and any combination thereof, with a combination of cetyl alcohol and stearyl alcohol being preferred.

The concentration of the fatty alcohol(s) preferably ranges between about 0.1 weight percentage and about 20 weight percentages of the total weight of the composition, more preferably it is between about 0.1 weight percentage and about 15 weight percentages, more preferably between about 0.1 weight percentage and about 12 weight percentages, more preferably between about 0.1 weight percentage and about 10 weight percentages, more preferably between about 0.1 weight percentage and about 5 weight percentages, more preferably between about 0.5 weight percentage and about 5 weight percentages, more preferably between about 0.5 weight percentage and about 2.5 weight percentages, and most preferably between 1.0 weight percentage and 2.0 weight percentages of the total weight of the composition.

As used herein, the phrase “surface-active agent” describes a chemical substance that has a lipophilic group and a hydrophilic group and therefore has the property of modifying the interfacial tension of the liquid in which it is dissolved. This phrase typically includes soaps, detergents, emulsifiers, dispersing agents and wetting agents.

Representative examples of surface active agents that are usable in the context of the present invention include, without limitation, polysorbate 60, ethoxylated sorbitan stearate, ethoxylated sorbitan palmitate, ethoxylated sorbitan oleate, nonyl phenol ethoxylates, fatty alcohol ethoxylates and any combinations thereof.

The preferred concentration of the surface-active agent(s) ranges between about 0.1 weight percentage and about 60 weight percentages of the total weight of the composition, more preferably between about 0.2 weight percentage and 30 weight percentages, more preferably between about 0.2 weight percentage and about 15 weight percentages, more preferably between about 0.2 weight percentage and about 10 weight percentages, more preferably between about 0.2 weight percentage and about 5 weight percentages, and most preferably between about 0.2 weight percentage and about 2.5 weight percentages of the total weight of the composition.

The concentration of the water in the composition of the present invention preferably ranges between about 10 weight percentages and about 40 weight percentages of the total weight of the composition, more preferably between about 20 weight percentages and about 40 weight percentages, more preferably between about 25 weight percentages and about 35 weight percentages, more preferably between about 28 weight percentages and about 32 weight percentages, and most preferably between about 30 weight percentages and about 32 weight percentages of the total weight of the composition.

As is discussed hereinabove, one of the major advantages of the composition of the present invention is the inclusion of a non-CFC propellant.

As used herein, the phrase “non-CFC propellant” describes a compound or a mixture of compounds characterized as propellants and not having a chemical composition that includes both a fluorine and chlorine atoms. The phrase “non-CFC propellant” therefore describes propellants that do not belong to the class of compounds known as chlorofluorocarbons (CFCs), which have been linked to the depletion of ozone in the atmosphere and are therefore considered environmentally hazardous.

Representative examples of non-CFC propellants that are usable in the context of the present invention include, without limitation, nitrous oxide, carbon dioxide, nitrogen, propane, iso-butane, n-butane, isopentane, n-pentane, dimethyl ether and any combination thereof.

It has been found that an exceptionally suitable non-CFC propellant comprises a mixture of propane, n-butane and isobutane.

The concentration of the non-CFC propellant in the composition of the present invention preferably ranges between about 1 weight percentage and about 40 weight percentages of the total weight of the composition, more preferably between about 1 weight percentage and about 20 weight percentages, more preferably between about 1 weight percentage and about 10 weight percentages, more preferably between about 1 weight percentage and about 8 weight percentages, more preferably between about 2 weight percentages and about 8 weight percentages, more preferably between about 3 weight percentages and about 6 weight percentages, with a concentration of about 4.5 weight percentages being the most preferred.

The foamable composition of the present invention is generally applied as a topical composition. As such, it is often advantageous to include at least one humectant in the composition. Representative examples of humectants that are usable in the context of the present invention include, without limitation, guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

The pH of the foamable composition of the present invention preferably ranges between about 4.0 and about 7.0. However, oftentimes it is desired to adjust the pH, so as to bring the pH into a preferred range (e.g., between 4.0 and 6.0) or to stabilize a component of the composition (vide infra).

However, the pH of a composition of the present invention is not modified nor preserved with the use of a buffer system, as is taught in the prior art, but rather with the addition of a pH-adjusting agent.

Representative examples of pH adjusting agents that are usable in the context of the present invention include, without limitation, adipic acid, glycine, calcium hydroxide, magnesium aluminometasilicates, hydrochloric acid, citric acid, phosphoric acid, lactic acid, sorbic acid and tartaric acid, and any combination thereof. Generally when a given pH-adjusting agent is included, the agent is the only source of a respective anion in the composition, such that no buffer system is included or produced in the composition.

In one preferred embodiment of the present invention, the carrier comprises ethanol, cetyl alcohol, stearyl alcohol, polysorbate 60 and water. According to a feature of this embodiment, the non-CFC propellant comprises a mixture of propane, n-butane and isobutane. According to another feature of this embodiment, the concentration of the non-CFC propellant ranges between about 1 weight percentage and about 40 weight percentages of the total weight of the composition.

In another preferred embodiment of the present invention, the concentration of the ethanol ranges between about 40 weight percentages and about 90 weight percentages of the composition, the concentration of the cetyl alcohol ranges between about 0.1 and about 20 weight percentages of the composition, the concentration of the stearyl alcohol ranges between about 0.1 and about 20 weight percentages of the composition, the concentration of the polysorbate 60 ranges between about 0.1 and about 60 weight percentages of the composition and the concentration of the water ranges between about 10 and about 40 weight percentages of the composition.

In yet another preferred embodiment of the present invention the concentration of the ethanol ranges between about 50 weight percentages and about 70 weight percentages of the composition, the concentration of the cetyl alcohol ranges between about 0.5 and about 10 weight percentages of the composition, the concentration of the stearyl alcohol ranges between about 0.4 and about 10 weight percentages of the composition, the concentration of the polysorbate 60 ranges between about 0.2 and about 15 weight percentages of the composition and the concentration of the water ranges between about 10 and about 40 weight percentages of the composition.

In still another preferred embodiment of the foamable composition of the present invention the concentration of the ethanol ranges between about 56 weight percentages and about 65 weight percentages of the composition, the concentration of the cetyl alcohol ranges between about 0.9 and about 1.3 weight percentages of the composition, the concentration of the stearyl alcohol ranges between about 0.4 and about 0.6 weight percentage of the composition, the concentration of the polysorbate 60 ranges between about 0.2 and about 0.6 weight percentage of the composition and the concentration of the water ranges between about 31 and about 36 weight percentages of the composition, and the composition further comprises propylene glycol, as a humectant, in a concentration of between about 1 and about 3 weight percentages of the composition, and a mixture of propane/butane/isobutene as a non-CFC propellant in a concentration of between about 4 and about 5 weight percentages of the composition, whereby the composition has a pH of about 6.5.

In another preferred embodiment of the foamable composition of the present invention the concentration of the ethanol ranges between about 56 weight percentages and about 65 weight percentages of the composition, the concentration of the cetyl alcohol ranges between about 0.9 and about 1.3 weight percentages of the composition, the concentration of the stearyl alcohol ranges between about 0.4 and about 0.6 weight percentage of the composition, the concentration of the polysorbate 60 ranges between about 0.2 and about 0.6 weight percentage of the composition and the concentration of the water ranges between about 31 and about 36 weight percentages of the composition, and the composition further comprises propylene glycol, as a humectant, in a concentration of between about 1 and about 3 weight percentages of the composition, and a mixture of propane/butane/isobutene as a non-CFC propellant in a concentration of between about 4 and about 5 weight percentages of the composition and lactic acid, whereby the concentration of the lactic acid is sufficient for adjusting the pH of the composition to between about 5.9 and about 6.1.

The pharmaceutical foamable composition of the present invention is particularly aimed at topically applying corticosteroids and comprises one or more corticosteroids as pharmaceutical active ingredients.

Representative examples of corticosteroids that are usable in the context of the present invention include, without limitation, clobetasol propionate, betamethasone valerate, alclometasone dipropionate, amcinonide, beclomethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone, fluprednidene acetate, flurandrenolone, fluticasone, halcinonide, hydrocortisone, hydrocortisone acetate, hydrocortisone butyrate, methylprednisolone acetate, mometasone furoate, triamcinolone acetonide, and any combination thereof, with betamethasone valerate and clobetasol propionate being preferred.

The preferred concentration of the corticosteroid active ingredient(s) in the foamable pharmaceutical composition of the present invention preferably ranges between about 0.01 weight percentage and about 1 weight percentage of the total weight of the composition, more preferably between about 0.02 weight percentages and about 1 weight percentage, more preferably between about 0.02 weight percentage and about 0.5 weight percentage, more preferably between about 0.02 weight percentage and about 0.1 weight percentage, more preferably between about 0.02 weight percentage and about 0.1 weight percentage, more preferably between about 0.02 weight percentages and about 0.08 weight percentage, more preferably between about 0.04 weight percentages and about 0.06 weight percentage, with a concentration of about 0.05 weight percentage being the most preferred.

As is discussed hereinabove, corticosteroids are pH sensitive compounds. For example, it is known that the preferred pH of compositions of clobetasol propionate and betamethasone valerate are about 6.0 and about 4.5, respectively, so as to prevent decomposition of these compounds. Such acidity is achieved and maintained using the teachings of the present invention, as is described hereinabove and is further demonstrated in the Examples section that follows.

The foamable pharmaceutical composition of the present invention can further include, in addition to the components described above, other ingredients such as, for example, antibacterial agents, bulking agents (e.g. mannitol), antioxidants (e.g., ascorbic acid or sodium bisulfite), anti-inflammatory agents, anti-viral agents, chemotherapeutic agents, anti-histamines and the like.

As corticosteroids are highly efficient pharmaceuticals, typically used in the treatment of various skin diseases and disorders, the foamable pharmaceutical composition of the present invention can be packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. Representative examples of a condition include, without limitation, acute inflammatory diseases, particularly skin diseases such as allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

Hence, according to another aspect of the present invention, there is provided a method of treatment, which is effected by administering, preferably topically or rectally, a therapeutically effective amount of the foamable pharmaceutical composition as described hereinabove to a mammal, especially a human, in need thereof.

A therapeutically (or pharmaceutically) effective amount, as used herein, means an amount of a corticosteroid needed to achieve the desired outcome, which is generally to prevent, alleviate or ameliorate the condition or symptoms of the condition described hereinabove. Determination of a therapeutically effective amount is within the capability of those skilled in the art, especially in light of the detailed disclosure provided herein.

The amount of a composition to be administered will, of course, be dependent on the subject being treated, the severity of the affliction, the manner of administration, the judgment of the prescribing physician, etc.

Generally, administering a composition of the present invention is effected by passing the composition from a first volume having a first pressure (e.g., a pressurized container) through a passage (e.g., a valve) into a volume having a second pressure, the second pressure being lower than the first pressure (e.g., the outside environment) so as to effect foaming of the composition. Preferably the foamable composition is administered onto a surface, such as skin.

The need for which the composition of the present invention is administered to a subject having a condition is generally a need such as curing the condition, treating the condition, preventing the condition, treating symptoms of the condition, curing symptoms of the condition, ameliorating symptoms of the condition, treating effects of the condition, ameliorating effects of the condition, and preventing results of the condition, whereby the condition is as described hereinabove.

Thus, according to the teachings above and as is further exemplified in the Examples section that follows, the carrier of the present invention, in combination with a non-CFC propellant, is exceptionally useful in implementing the innovative foamable pharmaceutical composition of the present invention for topical application of a corticosteroid active ingredient.

However, such a combination of the carrier of the present invention and a non-CFC propellant is also useful in implementing a general foamable composition, with or without an active ingredient.

Hence, according to another aspect of the present invention there is provided a foamable composition that comprises the non-CFC propellant and the carrier described hereinabove, which is devoid of a buffering agent.

Such a foamable composition can be used for applying any active ingredient, and, as it is characterized by maintaining the stability of ingredients that tend to decompose under inappropriate pH, it is particularly advantageous for applying a pH-sensitive active ingredients.

Therefore, a preferred foamable pharmaceutical composition according to this aspect of the present comprises one or more active ingredient(s), a non-CFC propellant and an acceptable carrier configured to generate a quick-break foam, as is described hereinabove, and is being devoid of a buffering agent. The active ingredient is preferably a pH-sensitive pharmaceutical active ingredient.

As used herein, the phrase “pH-sensitive pharmaceutical active ingredient” describes a pharmaceutically active ingredient that may be rendered unstable, e.g., may decompose, as a result of a change in the pH of the composition. pH sensitive active ingredients include, for example, compounds having a reactive functional group such as an ester group, which is susceptible for chemical reactions (e.g., de-esterification) under pH-dependent conditions.

Representative examples of pH-sensitive pharmaceutically active ingredients that can benefit from implementation within the composition of the present invention include corticosteroids, non-steroidal anti-inflammatory drugs, and the like.

A foamable composition according to this aspect of the present invention, which comprises a pharmaceutically active ingredient, can be packaged in a packaging material and identified in print, in or on the packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition. The condition can be, for example, an acute inflammatory disease, as is described hereinabove, or any other disease or disorder, preferably skin disease or disorder, that can be affected by the active ingredients described above.

According to a further aspect of the present invention, there is provided a process of preparing the foamable compositions described hereinabove. The process comprises obtaining a carrier by mixing at least one hydrocarbon alcohol, at least one fatty alcohol, at least one surface active agent, and water; placing the carrier in a pressure-resistant vessel; placing an amount of at least one non-CFC propellant into the pressure-resistant vessel; and sealing the pressure-resistant vessel.

According to a preferred embodiment of this aspect of the present invention, the process further comprises, prior to placing of the carrier in the vessel, admixing with the carrier one or more active ingredient(s), e.g., pH-sensitive pharmaceutically active ingredients, preferably, corticosteroids.

The process may further comprise, prior to placing the carrier in the vessel, admixing with the carrier at least one humectant.

According to an embodiment of this aspect of the present invention, the carrier is obtained by heating a mixture of the at least one hydrocarbon alcohol, the at least one fatty alcohol, the at least one surface-active agent and the water, at a temperature of at least 30° C., more preferably at least 40° C.

More particularly, the carrier can be obtained by mixing the water, the at least one fatty alcohol and the at least one surface-active agent, so as to obtain a clear aqueous solution; and thereafter adding the at least one hydrocarbon alcohol to the aqueous solution, to thereby obtain the carrier. The mixing is preferably performed while heating the aqueous solution at a temperature of at least about 40° C., preferably at least about 60° C. The addition of the hydrocarbon alcohol is preferably performed while heating the aqueous solution at a temperature of at least about 30° C., more preferably at a temperature of at least about 39° C.

Alternatively, the carrier can be obtained by mixing the hydrocarbon alcohol, the at least one fatty alcohol and the at least one surface-active agent, so as to obtain a clear alcoholic solution; and thereafter adding the water to the alcoholic solution, to thereby obtain the carrier. The addition of the water is preferably performed while heating the alcoholic solution at a temperature of at least about 30° C., preferably at a temperature of at least about 40° C.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description illustrate the invention in a non-limiting fashion.

Generally, the nomenclature used herein and the laboratory procedures utilized in the present invention include chemical and analytical techniques with which on skilled in the art is familiar. Unless otherwise defined, technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Preparation of Foamable Compositions:

Representative examples of the foamable compositions of the present invention were prepared using two processes, as described below:

Process A:

An alcoholic solution was prepared by combining indicated concentrations of stearyl alcohol, cetyl alcohol, polysorbate 60, propylene glycol and ethanol. The solution was heated to about 45° C. and was stirred until a clear solution was obtained. The clobetasol propionate was thereafter added and the obtained mixture was further stirred until a clear solution was obtained. Purified water was heated at a temperature of 45° C. and was thereafter added while mixing to the alcoholic phase. The combined solution was allowed to cool to room temperature.

The pH of the combined solution was adjusted, if necessary, by the addition of lactic acid to about 6.0.

The combined solution was poured into aerosol cans. A valve was attached to each can, the propellant was added and an actuator was assembled on the valve.

Process B:

An aqueous solution was prepared by combining the water, polysorbate 60 and the propylene glycol, heating the resulting solution to about 70° C. and stirring until a clear solution was obtained. The aqueous solution was thereafter cooled to 40° C. while stirring was continued.

The clobetasol propionate and the ethanol were mixed and the resulting solution was heated to 40° C. while stirring, until a clear alcohol solution was obtained. The alcohol solution and the aqueous solution were combined then while stirring, and the resulting solution was allowed to cool to room temperature.

The pH of the combined solution was adjusted at this stage, if necessary, by the addition of lactic acid to about 6.0.

The combined solution was poured into aerosol cans. A valve was attached to each can, the propellant was added and an actuator was assembled on the valve.

Using these processes, compositions I and II below were prepared.

Composition I

| | Ingredient | % (w/w) |
|---|---|---|
| 1. | Clobetasol propionate | 0.05 |
| 2. | Cetyl alcohol | 1.1 |
| 3. | Stearyl alcohol | 0.5 |
| 4. | Polysorbate 60 | 0.4 |
| 5. | Propylene glycol | 2.0 |
| 6. | Ethanol (96%) | 60.4 |
| 7. | Water | 31.05 |
| 8. | Propane/Butane/Isobutane | 4.5 |
| | Propellant | |

pH = 6.5

Composition II

| | Ingredient | % (w/w) |
|---|---|---|
| 1. | Clobetasol propionate | 0.05 |
| 2. | Cetyl alcohol | 1.1 |
| 3. | Stearyl alcohol | 0.5 |
| 4. | Polysorbate 60 | 0.4 |
| 5. | Propylene glycol | 2.0 |
| 6. | Ethanol (96%) | 60.4 |
| 7. | Water | 31.05 |
| 8. | Propane/Butane/Isobutane | 4.5 |
| | Propellant | |
| 9 | Lactic Acid | qs pH = 6.0 |

Performance:

From the four aerosol cans filled as described hereinabove, foam was sprayed in the usual way onto the skin of a testee. Excellent structured foam of median viscosity was produced and spread over a large area. The foam was observed to be quick-breaking.

Titration:

In order to ascertain that a composition of the present invention is not buffered despite the addition of a weak acid, the titration behavior of composition II (a) was compared to that of an identical composition devoid of lactic acid (b), and two samples of commercially available Olux® Foam containing 0.05% clobetasol propionate both having an identical expiry date more than a year from the time the stability evaluation was performed: batch # D3A003 manufactured by DPT Laboratories, Ltd. (San Antonio, Tex., USA) (c) and batch # 2E441 manufactured by CCL Pharmaceuticals (Cheshire, United Kingdom) (d).

Titration was performed in the usual way by titration with 0.1 N NaOH using a 682 Titroprocessor equipped with a 665 Dosimat and a 6.0233.100 glass electrode all by Metrohm Ltd. (Herisau, Switzerland). For direct titration, about 17 g of an analyte solution was transferred into 75 ml of purified water. For back titration, about 20 g of analyte solution was transferred into 510 ml of purified water and 0.1N HCl was added to adjust the pH to 2.0.

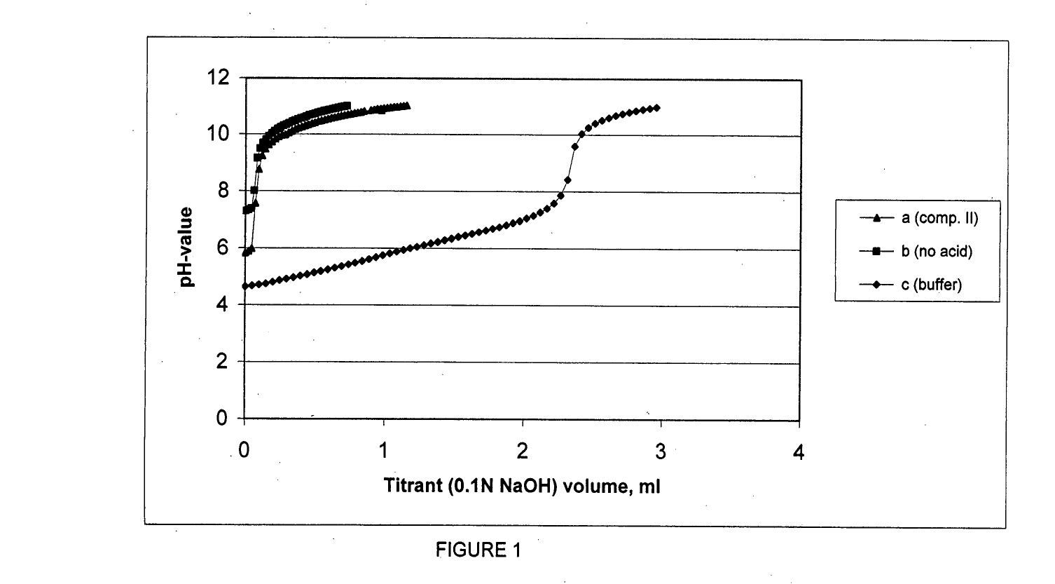

In FIG. 1, the results of direct titration of a clobetasol propionate composition acidified according to the teachings of the present invention (a), a non-acidifed clobetasol propionate composition having pH 7.0 (b) and a buffered prior art clobetasol propionate composition (c) are compared. From these results it is clear that the composition of the present invention is not buffered.

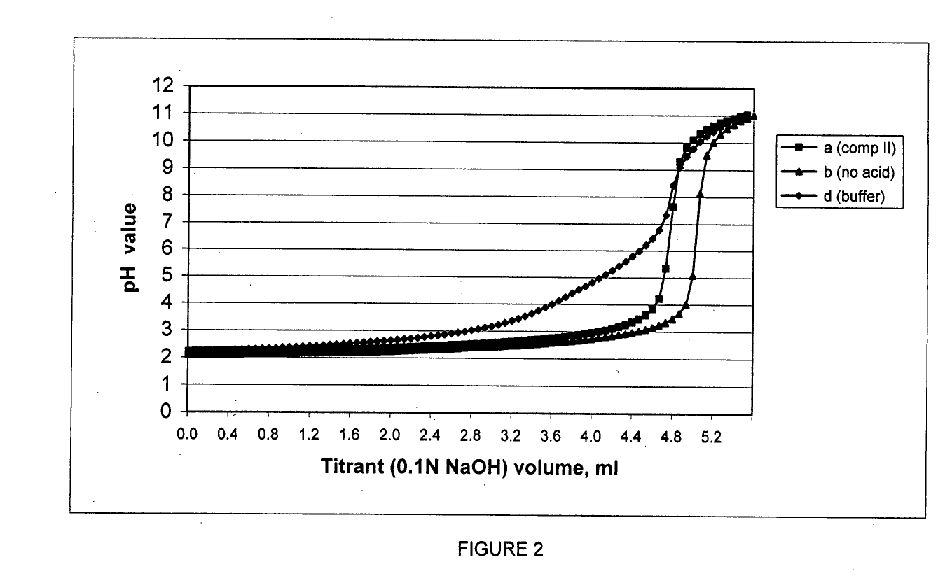

In FIG. 2, the results of back titration of a clobetasol propionate composition acidified according to the teachings of the present invention (a), a non-acidifed clobetasol propionate composition having pH 7.0 (b) and a buffered prior art clobetasol propionate composition (d) are compared. From these results it is clear that the composition of the present invention is not buffered.

Stability:

The relative stability of composition II (a) was evaluated by comparison to an identical composition devoid of lactic acid (b), and two samples of commercially available Olux® Foam containing 0.05% clobetasol propionate both having an identical expiry date more than a year from the time the stability evaluation was performed: (d) batch # 2E441 manufactured by CCL Pharmaceuticals, Ltd. (Cheshire, United Kingdom) and (e) batch # 2L741 manufactured by MIZA Pharmaceuticals (UK) Ltd. (Cheshire, United Kingdom).

All four samples were stored in identical aerosol cans at 40° C. and the stability measured at monthly intervals. Clobetasol propionate content and total degradant content were determined in the usual way using HPLC with a UV detector according to the USP Clobetasol propionate assay. The pH was determined in the usual way by titration with 0.1 N NaOH using a 682 Titroprocessor equipped with a 665 Dosimat and a 6.0233.100 glass electrode all by Metrohm Ltd. (Herisau, Switzerland).

The stability evaluation (summarized below in Table 1) demonstrates that the composition of the present invention (a) is more stable than the composition without a pH stabilizer (b) and either of the two prior art compositions (d) and (e).

TABLE 1
Relative stability of foamable clobetasol propionate compositions

| months | test | (a) | (b) | (d) | (e) |
|---|---|---|---|---|---|
| 0 | % clobetasol propionate | 0.053 | 0.052 | 0.051 | — |
| | % degradants | 0.17 | 0.15 | — | — |
| | pH | 6.1 | 6.7 | 6.1 | — |
| 1 | % clobetasol propionate | 0.053 | 0.051 | 0.048 | 0.048 |
| | % degradants | 0.36 | 0.36 | — | 2.16 |
| | pH | 5.9 | 6.1 | 6.2 | 6.2 |
| 2 | % clobetasol propionate | 0.054 | 0.05 | — | — |
| | % degradants | 0.35 | 0.55 | — | — |
| | pH | 5.7 | 5.9 | — | — |
| 3 | % clobetasol propionate | 0.050 | 0.048 | 0.046 | 0.046 |
| | % degradants | 1.75 | 1.65 | 3.22 | 3.28 |
| | pH | 5.9 | 6.0 | 6.3 | 6.2 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

1. A foamable pharmaceutical composition comprising at least one corticosteroid active ingredient, a non-CFC propellant and an acceptable carrier configured to generate a quick-break foam, the composition being devoid of a buffering agent.

2. The foamable pharmaceutical composition of claim 1, wherein said carrier comprises at least one hydrocarbon alcohol, at least one fatty alcohol, at least one surface active agent and water.

3. The foamable pharmaceutical composition of claim 2, wherein said at least one hydrocarbon alcohol has from one to ten carbon atoms.

4. The foamable pharmaceutical composition of claim 2, wherein said at least one hydrocarbon alcohol has from one to six carbon atoms.

5. The foamable pharmaceutical composition of claim 2, wherein said at least one hydrocarbon alcohol is an aliphatic hydrocarbon alcohol.

6. The foamable pharmaceutical composition of claim 5, wherein said aliphatic hydrocarbon alcohol is selected from the group consisting of methanol, ethanol, n-propanol, isopropanol, n-butanol, sec-butanol, isobutanol and t-butanol and mixtures thereof.

7. The foamable pharmaceutical composition of claim 2, wherein the concentration of said at least one hydrocarbon alcohol ranges between about 40 weight percentages and about 90 weight percentages of the total weight of the composition.

8. The foamable pharmaceutical composition of claim 2, wherein the concentration of said at least one hydrocarbon alcohol ranges between about 50 weight percentages and about 70 weight percentages of the total weight of the composition.

9. The foamable pharmaceutical composition of claim 2, wherein said at least one fatty alcohol has between 10 and 22 carbon atoms.

10. The foamable pharmaceutical composition of claim 9, wherein said at least one fatty alcohol is selected from the group consisting of cetyl alcohol, stearyl alcohol, lauryl alcohol, myristyl alcohol, palmityl alcohol and mixtures thereof.

11. The foamable pharmaceutical composition of claim 2, wherein the concentration of said at least one fatty alcohol ranges between about 0.1 weight percentage and about 20 weight percentages of the total weight of the composition.

12. The foamable pharmaceutical composition of claim 2, wherein the concentration of said at least one fatty alcohol ranges between about 0.5 weight percentage and about 10 weight percentages of the total weight of the composition.

13. The foamable pharmaceutical composition of claim 2, wherein the concentration of said water ranges between about 10 weight percentages and about 40 weight percentages of the total weight of the composition.

14. The foamable pharmaceutical composition of claim 2, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

15. The foamable pharmaceutical composition of claim 2, wherein the concentration of said at least one surface-active agent ranges between about 0.1 weight percentage and about 60 weight percentages of the total weight of the composition.

16. The foamable pharmaceutical composition of claim 2, wherein the concentration of said at least one surface-active agent ranges between about 0.2 weight percentage and about 15 weight percentages of the total weight of the composition.

17. The foamable pharmaceutical composition of claim 1, wherein the concentration of said non-CFC propellant ranges between about 1 weight percentage and about 40 weight percentages of the total weight of the composition.

18. The foamable pharmaceutical composition of claim 1, wherein the concentration of said non-CFC propellant ranges between about 1 weight percentage and about 20 weight percentages of the total weight of the composition.

19. The foamable pharmaceutical composition of claim 1, wherein said non-CFC propellant is selected from a group of propellants consisting of nitrous oxide, carbon dioxide, nitrogen, propane, iso-butane, n-butane, isopentane, n-pentane, dimethyl ether and any combination thereof.

20. The foamable pharmaceutical composition of claim 1, wherein said non-CFC propellant comprises a mixture of propane, n-butane and isobutane.

21. The foamable pharmaceutical composition of claim 1, further comprising at least one humectant.

22. The foamable pharmaceutical composition of claim 21, wherein said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

23. The foamable pharmaceutical composition of claim 1, further comprising at least one pH-adjusting agent.

24. The foamable pharmaceutical composition of claim 23, wherein said at least one pH adjusting agent is selected from the group consisting of adipic acid, glycine, calcium hydroxide, magnesium aluminometasilicates, hydrochloric acid, and any combination thereof.

25. The foamable pharmaceutical composition of claim 23, wherein said at least one pH adjusting agent is selected from the group of acids consisting of citric acid, phosphoric acid, lactic acid, sorbic acid and tartaric acid.

26. The foamable pharmaceutical composition of claim 25, wherein said acid is the only source of a respective anion in the composition.

27. The foamable pharmaceutical composition of claim 1, wherein said at least one corticosteroid active ingredient is selected from the group consisting of alclometasone dipropionate, amcinonide, beclomethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone, fluprednidene acetate, flurandrenolone, fluticasone, halcinonide, hydrocortisone, hydrocortisone acetate, hydrocortisone butyrate, methylprednisolone acetate, mometasone furoate, triamcinolone acetonide, and any combination thereof.

28. The foamable pharmaceutical composition of claim 1, wherein said at least one corticosteroid active ingredient is selected from the group consisting of betamethasone valerate and clobetasol propionate.

29. The foamable pharmaceutical composition of claim 1, wherein the concentration of said at least one corticosteroid active ingredient ranges between about 0.01 weight percentage and about 1 weight percentage of the total weight of the composition.

30. The foamable pharmaceutical composition of claim 1, wherein the concentration of said at least one corticosteroid active ingredient ranges between about 0.05 weight percentage and about 0.2 weight percentage of the total weight of the composition.

31. The foamable pharmaceutical composition of claim 1, having a pH of between about 4.0 and about 7.0.

32. The foamable pharmaceutical composition of claim 1, packaged in a packaging material and identified in print, in or on said packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition.

33. The foamable pharmaceutical composition of claim 32, wherein said condition is selected from the group consisting of acute inflammatory diseases, allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

34. The foamable pharmaceutical composition of claim 2, where said carrier comprises ethanol, cetyl alcohol, stearyl alcohol, polysorbate 60 and water.

35. The foamable pharmaceutical composition of claim 34, wherein the concentration of said ethanol ranges between about 40 weight percentages and about 90 weight percentages of the composition, the concentration of said cetyl alcohol ranges between about 0.1 and about 20 weight percentages of the composition, the concentration of said stearyl alcohol ranges between about 0.1 and about 20 weight percentages of the composition, the concentration of said polysorbate 60 ranges between about 0.1 and about 60 weight percentages of the composition and the concentration of said water ranges between about 10 and about 40 weight percentages of the composition.

36. The foamable pharmaceutical composition of claim 34, wherein the concentration of said ethanol ranges between about 50 weight percentages and about 70 weight percentages of the composition, the concentration of said cetyl alcohol ranges between about 0.5 and about 10 weight percentages of the composition, the concentration of said stearyl alcohol ranges between about 0.4 and about 10 weight percentages of the composition, the concentration of said polysorbate 60 ranges between about 0.2 and about 15 weight percentages of the composition and the concentration of said water ranges between about 10 and about 40 weight percentages of the composition.

37. The foamable pharmaceutical composition of claim 34, wherein said non-CFC propellant comprises a mixture of propane, n-butane and isobutane.

38. The foamable pharmaceutical composition of claim 37, wherein the concentration of said non-CFC propellant ranges between about 1 weight percentage and about 40 weight percentages of the total weight of the composition.

39. The foamable pharmaceutical composition of claim 34, wherein said at least one corticosteroid active ingredient is selected from the group consisting of clobetasol propionate and betamethasone valerate.

40. The foamable pharmaceutical composition of claim 39, wherein the concentration of said at least one corticosteroid active ingredient ranges between about 0.01 weight percentage and about 1 weight percentage of the total weight of the composition.

41. The foamable pharmaceutical composition of claim 36, wherein the concentration of said ethanol ranges between about 56 weight percentages and about 65 weight percentages of the composition, the concentration of said cetyl alcohol ranges between about 0.9 and about 1.3 weight percentages of the composition, the concentration of said stearyl alcohol ranges between about 0.4 and about 0.6 weight percentage of the composition, the concentration of said polysorbate 60 ranges between about 0.2 and about 0.6 weight percentage of the composition and the concentration of said water ranges between about 31 and about 36 weight percentages of the composition and further comprising propylene glycol in a concentration of between about 1 and about 3 weight percentages of the composition, propane/butane/isobutene as a non-CFC propellant in a concentration of between about 4 and about 5 weight percentages of the composition, and clobetasol propionate in a concentration of between about 0.01 and about 0.1 weight percentage of the composition, the composition having a pH of about 6.5.

42. The foamable pharmaceutical composition of claim 36, wherein the concentration of said ethanol ranges between about 56 weight percentages and about 65 weight percentages of the composition, the concentration of said cetyl alcohol ranges between about 0.9 and about 1.3 weight percentages of the composition, the concentration of said stearyl alcohol ranges between about 0.4 and about 0.6 weight percentage of the composition, the concentration of said polysorbate 60 ranges between about 0.2 and about 0.6 weight percentage of the composition and the concentration of said water ranges between about 31 and about 36 weight percentages of the composition and further comprising propylene glycol in a concentration of between about 1 and about 3 weight percentages of the composition, propane/butane/isobutane as a non-CFC propellant in a concentration of between about 4 and about 5 weight percentages of the composition, and clobetasol propionate in a concentration of between about 0.01 and about 0.1 weight percentage of the composition and lactic acid, whereby the concentration of said lactic acid is sufficient for adjusting the pH of the composition to between about 5.9 and about 6.1.

43. A method of treatment comprising administering a therapeutically effective amount of a foamable pharmaceutical composition of claim 1 to a mammal in need thereof.

44. The method of claim 43, wherein said mammal is a human.

45. The method of claim 43, wherein said administering is effected topically.

46. The method of claim 45, wherein said administering comprises:
passing said composition from a first volume having a first pressure through a passage into a volume having a second pressure so as to effect foaming of said composition,
wherein said first pressure is greater than said second pressure.

47. The method of claim 46, further comprising applying said foamable composition onto a surface.

48. The method of claim 47, wherein said surface is skin.

49. The method of claim 43, wherein said need arises from a medical condition selected from the group consisting of acute inflammatory diseases, allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

50. The method of claim 43, wherein said need is selected from the group consisting of curing said condition, treating said condition, preventing said condition, treating symptoms of said condition, curing symptoms of said condition, ameliorating symptoms of said condition, treating effects of said condition, ameliorating effects of said condition, and preventing results of said condition.

51. The method of claim 43, wherein said at least one corticosteroid active ingredient is selected from the group consisting of alclometasone dipropionate, amcinonide, beclomethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone, fluprednidene acetate, flurandrenolone, fluticasone, halcinonide, hydrocortisone, hydrocortisone acetate, hydrocortisone butyrate, methylprednisolone acetate, mometasone furoate, triamcinolone acetonide, and mixtures thereof.

52. The method of claim 43, wherein said at least one corticosteroid active ingredient is selected from the group consisting of betamethasone valerate and clobetasol propionate.

53. The method of claim 43, wherein said carrier comprises at least one hydrocarbon alcohol, at least one fatty alcohol, at least one surface active agent and water.

54. The method of claim 53, wherein said at least one hydrocarbon alcohol has from one to ten carbon atoms.

55. The method of claim 53, wherein said at least one hydrocarbon alcohol has from one to six carbon atoms.

56. The method of claim 53, wherein said at least one hydrocarbon alcohol is an aliphatic hydrocarbon alcohol.

57. The method of claim 53, wherein said aliphatic hydrocarbon alcohol is selected from the group consisting of methanol, ethanol, n-propanol, isopropanol, n-butanol, sec-butanol, isobutanol and t-butanol and mixtures thereof.

58. The method of claim 53, wherein the concentration of said at least one hydrocarbon alcohol ranges between about 40 weight percentages and about 90 weight percentages of the total weight of said composition.

59. The method of claim 53, wherein the concentration of said at least one hydrocarbon alcohol ranges between about 50 weight percentages and about 70 weight percentages of the total weight of said composition.

60. The method of claim 53, wherein said at least one fatty alcohol has between 10 and 22 carbon atoms.

61. The method of claim 60, wherein said at least one fatty alcohol is selected from the group consisting of cetyl alcohol, stearyl alcohol, lauryl alcohol, myristyl alcohol, palmityl alcohol and mixtures thereof.

62. The method of claim 53, wherein the concentration of said at least one fatty alcohol ranges between about 0.1 weight percentage and about 20 weight percentages of the total weight of said composition.

63. The method of claim 53, wherein the concentration of said water ranges between about 0.5 weight percentage and about 10 weight percentages of the total weight of said composition.

64. The method of claim 53, wherein the concentration of said water ranges between about 10 weight percentages and about 40 weight percentages of the total weight of said composition.

65. The method of claim 53, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

66. The method of claim 53, wherein the concentration of said at least one surface-active agent ranges between about 0.1 weight percentage and about 60 weight percentages of the total weight of said composition.

67. The method of claim 53, wherein the concentration of said at least one surface-active agent ranges between about 0.2 weight percentage and about 15 weight percentages of the total weight of said composition.

68. The method of claim 43, wherein the concentration of said non-CFC propellant ranges between about 1 weight percentage and about 40 weight percentages of the total weight of said composition.

69. The method of claim 43, wherein the concentration of said non-CFC propellant ranges between about 1 weight percentage and about 20 weight percentages of the total weight of said composition.

70. The method of claim 43, wherein said non-CFC propellant is selected from a group of propellants consisting of nitrous oxide, carbon dioxide, nitrogen, propane, iso-butane, n-butane, isopentane, n-pentane, dimethyl ether and any combination thereof.

71. The method of claim 43, wherein said non-CFC propellant comprises a mixture of propane, n-butane and isobutane.

72. The method of claim 43, wherein said foamable pharmaceutical composition further comprises at least one humectant.

73. The method of claim 72, wherein said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

74. The method of claim 43, wherein said foamable pharmaceutical composition further comprises at least one pH-adjusting agent.

75. The method of claim 74, wherein said at least one pH adjusting agent is selected from the group consisting of adipic acid, glycine, calcium hydroxide, magnesium aluminometasilicates, hydrochloric acid, and any combination thereof.

76. The method of claim 74, wherein said at least one pH adjusting agent is selected from the group of acids consisting of citric acid, phosphoric acid, lactic acid, sorbic acid and tartaric acid.

77. The method of claim 76, wherein said acid is the only source of a respective anion in said composition.

78. The method of claim 43, wherein the concentration of said at least one corticosteroid active ingredient ranges between about 0.01 weight percentage and about 1 weight percentage of the total weight of said composition.

79. The method of claim 43, wherein the concentration of said at least one corticosteroid active ingredient of said carrier ranges between about 0.05 weight percentage and about 0.2 weight percentage of the total weight of said composition.

80. The method of claim 53, wherein said carrier has a pH of between about 4.0 and about 7.0.

81. The method of claim 53, wherein said carrier comprises ethanol, cetyl alcohol, stearyl alcohol, polysorbate 60 and water.

82. The method of claim 81, wherein said ethanol ranges in a concentration of between about 40 weight percentages and about 90 weight percentages of said composition, said cetyl alcohol ranges in a concentration of between about 0.1 and about 20 weight percentages of said composition, said stearyl alcohol ranges in a composition of between about 0.1 and about 20 weight percentages of said composition, said polysorbate 60 ranges in a concentration of between about 0.1 and about 60 weight percentages of said composition and said water ranges in a concentration of between about 10 and about 40 weight percentages of said composition.

83. The method of claim 81, wherein said ethanol ranges in a concentration of between about 50 weight percentages and about 70 weight percentages of said composition, said cetyl alcohol ranges in a concentration of between about 0.5 and about 10 weight percentages of said composition, said stearyl alcohol ranges in a composition of between about 0.4 and about 10 weight percentages of said composition, said polysorbate 60 ranges in a concentration of between about 0.2 and about 15 weight percentages of said composition and said water ranges in a concentration of between about 10 and about 40 weight percentages of said composition.

84. The method of claim 81, wherein said non-CFC propellant comprises a mixture of propane, n-butane and isobutane.

85. The method of claim 84, wherein said the concentration of said non-CFC propellant ranges between about 1 weight percentage and about 40 weight percentages of the total weight of said composition.

86. The method of claim 81, wherein said at least one corticosteroid active ingredient is selected from the group consisting of clobetasol propionate and betamethasone valerate.

87. The method of claim 86, wherein the concentration of said at least one corticosteroid active ingredient ranges between about 0.01 weight percentage and about 1 weight percentage of the total weight of said composition.

88. The method of claim 83, wherein the concentration of said ethanol ranges between about 56 weight percentages and about 65 weight percentages of said composition, the concentration of said cetyl alcohol ranges between about 0.9 and about 1.3 weight percentages of said composition, the concentration of said stearyl alcohol ranges between about 0.4 and about 0.6 weight percentage of said composition, the concentration of said polysorbate 60 ranges between about 0.2 and about 0.6 weight percentage of said composition and the concentration of said water ranges between about 31 and about 36 weight percentages of said composition and further comprising propylene glycol in a concentration of between about 1 and about 3 weight percentages of said composition, propane/butane/isobutene as a non-CFC propellant in a concentration of between about 4 and about 5 weight percentages of said composition, and clobetasol propionate in a concentration of between about 0.01 and about 0.1 weight percentage of said composition, the composition having pH of about 6.5.

89. The method of claim 83, wherein the concentration of said ethanol ranges between about 56 weight percentages and about 65 weight percentages of said composition, the concentration of said cetyl alcohol ranges between about 0.9 and about 1.3 weight percentages of said composition, the concentration of said stearyl alcohol ranges between about 0.4 and about 0.6 weight percentage of said composition, the concentration of said polysorbate 60 ranges between about 0.2 and about 0.6 weight percentage of said composition and the concentration of said water ranges between about 31 and about 36 weight percentages of said composition and further comprising propylene glycol in a concentration of between about 1 and about 3 weight percentages of said composition, propane/butane/isobutane as a non-CFC propellant in a concentration of between about 4 and about 5 weight percentages of said composition, and clobetasol propionate in a concentration of between about 0.01 and about 0.1 weight percentage of said composition and lactic acid, whereby the concentration of said lactic acid is sufficient for adjusting the pH of said composition to between about 5.9 and about 6.1.

90. A process of preparing a foamable composition of claim 1, the process comprising:
obtaining said carrier by mixing at least one hydrocarbon alcohol, at least one fatty alcohol, at least one surface active agent, and water;
placing said carrier in a pressure-resistant vessel;
placing an amount of at least one non-CFC propellant into said pressure-resistant vessel; and
sealing said pressure-resistant vessel.

91. The process of claim 90, further comprising, prior to said placing:
admixing with said carrier said corticosteroid active ingredient.

92. The process of claim 90, further comprising, prior to said placing:
admixing with said carrier at least one humectant.

93. The process of claim 92, where said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

94. The process of claim 90, wherein said obtaining includes heating a mixture of said at least one hydrocarbon alcohol, said at least one fatty alcohol, said at least one surface active agent and said water, at a temperature of at least 30° C.

95. The process of claim 90, wherein said obtaining includes heating a mixture of said at least one hydrocarbon alcohol, said at least one fatty alcohol, said at least one surface active agent and said water, at a temperature of at least 40° C.

96. The process of claim 90, wherein said at least one hydrocarbon alcohol comprises at least one aliphatic alcohol having from 1 to 6 carbon atoms.

97. The process of claim 96, wherein said at least one aliphatic alcohol is selected from the group consisting of methanol, ethanol, n-propanol, isopropanol, n-butanol, sec-butanol, isobutanol and t-butanol and mixtures thereof.

98. The process of claim 90, wherein said at least one fatty alcohol has between 10 and 22 carbon atoms.

99. The process of claim 98, wherein said at least one fatty alcohol is selected from the group consisting of cetyl alcohol, stearyl alcohol, lauryl alcohol, myristyl alcohol, palmityl alcohol and mixtures thereof.

100. The process of claim 90, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

101. The process of claim 90, wherein said non-CFC propellant is selected from a group of propellants consisting of nitrous oxide, carbon dioxide, nitrogen, propane, iso-butane, n-butane, isopentane, n-pentane, dimethyl ether and mixtures thereof.

102. The process of claim 90, wherein said obtaining comprises:
mixing said water, said at least one fatty alcohol and said at least one surface active agent, so as to obtain a clear aqueous solution; and
adding said at least one hydrocarbon alcohol to said aqueous solution, to thereby obtain said carrier.

103. The process of claim 102, wherein said mixing further includes heating said aqueous solution at a temperature of at least about 40° C.

104. The process of claim 102, wherein said mixing further includes heating said aqueous solution at a temperature of at least about 60° C.

105. The process of claim 102, wherein said adding is performed while heating said aqueous solution at a temperature of at least about 30° C.

106. The process of claim 102, wherein said adding is performed while heating said aqueous solution at a temperature of at least about 39° C.

107. The process of claim 90, wherein said obtaining comprises:
mixing said hydrocarbon alcohol, said at least one fatty alcohol and said at least one surface active agent, so as to obtain a clear alcoholic solution; and
adding said water to said alcoholic solution, to thereby obtain said carrier.

108. The process of claim 107, wherein said adding is performed while heating said alcoholic solution at a temperature of at least about 30° C.

109. The process of claim 107, said adding is performed while heating said alcoholic solution at a temperature of at least about 40° C.

110. A foamable composition comprising a non-CFC propellant and a carrier configured to generate a quick-break foam, the composition being devoid of a buffering agent.

111. The foamable composition of claim 110, further comprising at least one pharmaceutically active ingredient.

112. The foamable composition of claim 111, wherein said at least one pharmaceutical active ingredient is a pH sensitive pharmaceutical active ingredient.

113. The foamable composition of claim 112, wherein said at least one pharmaceutically active ingredient is selected from the group consisting of corticosteroids and non-steroidal anti-inflammatory drugs.

114. The foamable composition of claim 110, wherein said carrier comprises at least one hydrocarbon alcohol, at least one fatty alcohol, at least one surface active agent and water.

115. The foamable composition of claim 114, wherein said at least one hydrocarbon alcohol has from one to ten carbon atoms.

116. The foamable composition of claim 114, wherein said at least one hydrocarbon alcohol has from one to six carbon atoms.

117. The foamable composition of claim 114, wherein said at least one hydrocarbon alcohol is an aliphatic hydrocarbon alcohol.

118. The foamable composition of claim 117, wherein said aliphatic hydrocarbon alcohol is selected from the group consisting of methanol, ethanol, n-propanol, isopropanol, n-butanol, sec-butanol, isobutanol and t-butanol and mixtures thereof.

119. The foamable composition of claim 114, wherein the concentration of said at least one hydrocarbon alcohol ranges between about 40 weight percentages and about 90 weight percentages of the total weight of the composition.

120. The foamable composition of claim 114, wherein the concentration of said at least one hydrocarbon alcohol ranges between about 50 weight percentages and about 70 weight percentages of the total weight of the composition.

121. The foamable composition of claim 114, wherein said at least one fatty alcohol has between 10 and 22 carbon atoms.

122. The foamable composition of claim 121, wherein said at least one fatty alcohol is selected from the group consisting of cetyl alcohol, stearyl alcohol, lauryl alcohol, myristyl alcohol, palmityl alcohol and mixtures thereof.

123. The foamable composition of claim 114, wherein the concentration of said at least one fatty alcohol ranges between about 0.1 weight percentage and about 20 weight percentages of the total weight of the composition.

124. The foamable composition of claim 114, wherein the concentration of said at least one fatty alcohol ranges between about 0.5 weight percentage and about 10 weight percentages of the total weight of the composition.

125. The foamable composition of claim 114, wherein the concentration of said water ranges between about 10 weight percentages and about 40 weight percentages of the total weight of the composition.

126. The foamable composition of claim 114, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

127. The foamable composition of claim 114, wherein the concentration of said at least one surface-active agent ranges between about 0.1 weight percentage and about 60 weight percentages of the total weight of the composition.

128. The foamable composition of claim 114, wherein the concentration of said at least one surface-active agent ranges between about 0.2 weight percentage and about 15 weight percentages of the total weight of the composition.

129. The foamable composition of claim 110, wherein the concentration of said non-CFC propellant ranges between about 1 weight percentage and about 40 weight percentages of the total weight of the composition.

130. The foamable composition of claim 110, wherein the concentration of said non-CFC propellant ranges between about 1 weight percentage and about 20 weight percentages of the total weight of the composition.

131. The foamable composition of claim 110, wherein said non-CFC propellant is selected from a group of propellants consisting of nitrous oxide, carbon dioxide, nitrogen, propane, iso-butane, n-butane, isopentane, n-pentane, dimethyl ether and any combination thereof.

132. The foamable composition of claim 110, wherein said non-CFC propellant comprises a mixture of propane, n-butane and isobutane.

133. The foamable composition of claim 110, further comprising at least one humectant.

134. The foamable composition of claim 133, wherein said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

135. The foamable composition of claim 110, further comprising at least one pH adjusting agent.

136. The foamable composition of claim 135, wherein said at least one pH adjusting agent is selected from the group consisting of adipic acid, glycine, calcium hydroxide, magnesium aluminometasilicates, hydrochloric acid, and any combination thereof.

137. The foamable composition of claim 135, wherein said at least one pH adjusting agent is selected from the group of acids consisting of citric acid, phosphoric acid, lactic acid, sorbic acid and tartaric acid.

138. The foamable pharmaceutical composition of claim 137, wherein said acid is the only source of a respective anion in the composition.

139. The foamable composition of claim 110, having a pH of between about 4.0 and about 7.0.

140. The foamable composition of claim 110, packaged in a packaging material and identified in print, in or on said packaging material, for use for a need selected from the group consisting of curing a condition, treating a condition, preventing a condition, treating symptoms of a condition, curing symptoms of a condition, ameliorating symptoms of a condition, treating effects of a condition, ameliorating effects of a condition, and preventing results of a condition.

141. The foamable composition of claim 114, where said carrier comprises ethanol, cetyl alcohol, stearyl alcohol, polysorbate 60 and water.

142. The foamable composition of claim 141, wherein the concentration of said ethanol ranges between about 40 weight percentages and about 90 weight percentages of the composition, the concentration of said cetyl alcohol ranges between about 0.1 and about 20 weight percentages of the composition, the concentration of said stearyl alcohol ranges between about 0.1 and about 20 weight percentages of the composition, the concentration of said polysorbate 60 ranges between about 0.1 and about 60 weight percentages of the composition and the concentration of said water ranges between about 10 and about 40 weight percentages of the composition.

143. The foamable composition of claim 141, wherein the concentration of said ethanol ranges between about 50 weight percentages and about 70 weight percentages of the composition, the concentration of said cetyl alcohol ranges between about 0.1 and about 10 weight percentages of the composition, the concentration of said stearyl alcohol ranges between about 0.4 and about 10 weight percentages of the composition, the concentration of said polysorbate 60 ranges between about 0.2 and about 15 weight percentages of the composition and the concentration of said water ranges between about 10 and about 40 weight percentages of the composition.

144. The foamable composition of claim 141, wherein said non-CFC propellant comprises a mixture of propane, n-butane and isobutane.

145. The foamable composition of claim 144, wherein the concentration of said non-CFC propellant ranges between about 1 weight percentage and about 40 weight percentages of the total weight of the composition.

146. The foamable composition of claim 143, wherein the concentration of said ethanol ranges between about 56 weight percentages and about 65 weight percentages of the composition, the concentration of said cetyl alcohol ranges between about 0.9 and about 1.3 weight percentages of the composition, the concentration of said stearyl alcohol ranges between about 0.4 and about 0.6 weight percentage of the composition, the concentration of said polysorbate 60 ranges between about 0.2 and about 0.6 weight percentage of the composition and the concentration of said water ranges between about 31 and about 36 weight percentages of the composition and further comprising propylene glycol in a concentration of between about 1 and about 3 weight percentages of the composition, propane/butane/isobutene as a non-CFC propellant in a concentration of between about 4 and about 5 weight percentages of the composition, the composition having pH of about 6.5.

147. The foamable composition of claim 143, wherein the concentration of said ethanol ranges between about 56 weight percentages and about 65 weight percentages of the composition, the concentration of said cetyl alcohol ranges between about 0.9 and about 1.3 weight percentages of the composition, the concentration of said stearyl alcohol ranges between about 0.4 and about 0.6 weight percentage of the composition, the concentration of said polysorbate 60 ranges between about 0.2 and about 0.6 weight percentage of the composition and the concentration of said water ranges between about 31 and about 36 weight percentages of the composition and further comprising propylene glycol in a concentration of between about 1 and about 3 weight percentages of the composition, propane/butane/isobutane as a non-CFC propellant in a concentration of between about 4 and about 5 weight percentages of the composition and lactic acid, whereby the concentration of said lactic acid is sufficient for adjusting the pH of the composition to between about 5.9 and about 6.1.

148. A method of treatment comprising administering an amount of a foamable composition of claim 111 to a mammal in need thereof.

149. The method of claim 148, wherein said mammal is a human.

150. The method of claim 148, wherein said administering is effected topically.

151. The method of claim 150, wherein said administering comprises:
passing said composition from a first volume having a first pressure through a passage into a volume having a second pressure so as to effect foaming of said composition,
wherein said first pressure is greater than said second pressure.

152. The method of claim 151, further comprising applying said foamable composition onto a surface.

153. The method of claim 152, wherein said surface is skin.

154. The method of claim 112, wherein said at least one pharmaceutically active ingredient is a corticosteroid and said need arises from a medical condition selected from the group consisting of acute inflammatory diseases, allergic contact dermatitis, eczema, atopic eczema, asteatotic eczema, discoid eczema, infantile eczema and napkin dermatitis, psoriasis—plaque, seborrheic dermatitis, atopic dermatitis, dermatitis herpetiformis, neurodermatitis, lichen simplex chronicus, lichen planus, subacute cutaneous lupus erythematosus, papular urticaria, palmoplantar psoriasis, discoid lupus erythematosus, chronic hypertrophic lichen planus, granuloma annulare and keloid scars.

155. The method of claim 148, wherein said need is selected from the group consisting of curing said condition, treating said condition, preventing said condition, treating symptoms of said condition, curing symptoms of said condition, ameliorating symptoms of said condition, treating effects of said condition, ameliorating effects of said condition, and preventing results of said condition.

156. The method of claim 148, wherein said carrier comprises at least one hydrocarbon alcohol, at least one fatty alcohol, at least one surface active agent and water.

157. The method of claim 156, wherein said at least one hydrocarbon alcohol has from one to ten carbon atoms.

158. The method of claim 156, wherein said at least one hydrocarbon alcohol has from one to six carbon atoms.

159. The method of claim 156, wherein said at least one hydrocarbon alcohol is an aliphatic hydrocarbon alcohol.

160. The method of claim 156, wherein said aliphatic hydrocarbon alcohol is selected from the group consisting of methanol, ethanol, n-propanol, isopropanol, n-butanol, sec-butanol, isobutanol and t-butanol and mixtures thereof.

161. The method of claim 156, wherein the concentration of said at least one hydrocarbon alcohol ranges between about 40 weight percentages and about 90 weight percentages of the total weight of said composition.

162. The method of claim 156, wherein the concentration of said at least one hydrocarbon alcohol ranges between about 50 weight percentages and about 70 weight percentages of the total weight of said composition.

163. The method of claim 156, wherein said at least one fatty alcohol has between 10 and 22 carbon atoms.

164. The method of claim 163, wherein said at least one fatty alcohol is selected from the group consisting of cetyl alcohol, stearyl alcohol, lauryl alcohol, myristyl alcohol, palmityl alcohol and mixtures thereof.

165. The method of claim 156, wherein the concentration of said at least one fatty alcohol ranges between about 0.1 weight percentage and about 20 weight percentages of the total weight of said composition.

166. The method of claim 156, wherein the concentration of said water ranges between about 0.5 weight percentage and about 10 weight percentages of the total weight of said composition.

167. The method of claim 156, wherein the concentration of said water ranges between about 10 weight percentages and about 40 weight percentages of the total weight of said composition.

168. The method of claim 156, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

169. The method of claim 156, wherein the concentration of said at least one surface-active agent ranges between about 0.1 weight percentage and about 60 weight percentages of the total weight of said composition.

170. The method of claim 156, wherein the concentration of said at least one surface-active agent ranges between about 0.2 weight percentage and about 15 weight percentages of the total weight of said composition.

171. The method of claim 148, wherein the concentration of said non-CFC propellant ranges between about 1 weight percentage and about 40 weight percentages of the total weight of said composition.

172. The method of claim 148, wherein the concentration of said non-CFC propellant ranges between about 1 weight percentage and about 20 weight percentages of the total weight of said composition.

173. The method of claim 148, wherein said non-CFC propellant is selected from a group of propellants consisting of nitrous oxide, carbon dioxide, nitrogen, propane, iso-butane, n-butane, isopentane, n-pentane, dimethyl ether and any combination thereof.

174. The method of claim 148, wherein said non-CFC propellant comprises a mixture of propane, n-butane and isobutane.

175. The method of claim 148, wherein said foamable composition further comprises at least one humectant.

176. The method of claim 175, wherein said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

177. The method of claim 148, wherein said foamable composition further comprises at least one pH-adjusting agent.

178. The method of claim 177, wherein said at least one pH adjusting agent is selected from the group consisting of adipic acid, glycine, calcium hydroxide, magnesium aluminometasilicates, hydrochloric acid, and any combination thereof.

179. The method of claim 177, wherein said at least one pH adjusting agent is selected from the group of acids consisting of citric acid, phosphoric acid, lactic acid, sorbic acid and tartaric acid.

180. The method of claim 179, wherein said acid is the only source of a respective anion in said composition.

181. The method of claim 156, wherein said carrier has a pH of between about 4.0 and about 7.0.

182. The method of claim 156, wherein said carrier comprises ethanol, cetyl alcohol, stearyl alcohol, polysorbate 60 and water.

183. The method of claim 182, wherein said ethanol ranges in a concentration of between about 40 weight percentages and about 90 weight percentages of said composition, said cetyl alcohol ranges in a concentration of between about 0.1 and about 20 weight percentages of said composition, said stearyl alcohol ranges in a composition of between about 0.1 and about 20 weight percentages of said composition, said polysorbate 60 ranges in a concentration of between about 0.1 and about 60 weight percentages of said composition and said water ranges in a concentration of between about 10 and about 40 weight percentages of said composition.

184. The method of claim 182 wherein said ethanol ranges in a concentration of between about 50 weight percentages and about 70 weight percentages of said composition, said cetyl alcohol ranges in a concentration of between about 0.5 and about 10 weight percentages of said composition, said stearyl alcohol ranges in a composition of between about 0.4 and about 10 weight percentages of said composition, said polysorbate 60 ranges in a concentration of between about 0.2 and about 15 weight percentages of said composition and said water ranges in a concentration of between about 10 and about 40 weight percentages of said composition.

185. The method of claim 182, wherein said non-CFC propellant comprises a mixture of propane, n-butane and isobutane.

186. The method of claim 185, wherein said the concentration of said non-CFC propellant ranges between about 1 weight percentage and about 40 weight percentages of the total weight of said composition.

187. The method of claim 184, wherein the concentration of said ethanol ranges between about 56 weight percentages and about 65 weight percentages of said composition, the concentration of said cetyl alcohol ranges between about 0.9 and about 1.3 weight percentages of said composition, the concentration of said stearyl alcohol ranges between about 0.4 and about 0.6 weight percentage of said composition, the concentration of said polysorbate 60 ranges between about 0.2 and about 0.6 weight percentage of said composition and the concentration of said water ranges between about 31 and about 36 weight percentages of said composition and further comprising propylene glycol in a concentration of between about 1 and about 3 weight percentages of said composition, propane/butane/isobutane as a non-CFC propellant in a concentration of between about 4 and about 5 weight percentages of said composition, the composition having pH of about 6.5.

188. The method of claim 184, wherein the concentration of said ethanol ranges between about 56 weight percentages and about 60 weight percentages of said composition, the concentration of said cetyl alcohol ranges between about 0.9 and about 1.3 weight percentages of said composition, the concentration of said stearyl alcohol ranges between about 0.4 and about 0.6 weight percentage of said composition, the concentration of said polysorbate 60 ranges between about 0.2 and about 0.6 weight percentage of said composition and the concentration of said water ranges between about 31 and about 36 weight percentages of said composition and further comprising propylene glycol in a concentration of between about 1 and about 3 weight percentages of said composition, propanelbutane/isobutane as a non-CFC propellant in a concentration of between about 4 and about 5 weight percentages of said composition, and lactic acid, whereby the concentration of said lactic acid is sufficient for adjusting the pH of said composition to between about 5.9 and about 6.1.

189. A process of preparing the foamable composition of claim 110, the process comprising:
obtaining said carrier by combining at least one hydrocarbon alcohol, at least one fatty alcohol, at least one surface active agent, and water;
placing said carrier in a pressure-resistant vessel;
placing an amount of at least one non-CFC propellant into said pressure resistant vessel; and
sealing said pressure-resistant vessel.

190. The process of claim 189, further comprising, prior to said placing:
admixing with said carrier at least one humectant.

191. The process of claim 190, wherein said at least one humectant is selected from the group consisting of guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, polyhydroxy alcohol, sorbitol, glycerol, hexanetriol, propylene glycol, butylene glycol, hexylene glycol, a hexylene glycol derivative, polyethylene glycol, a sugar, a starch, a sugar derivative, a starch derivative, alkoxylated glucose, hyaluronic acid, lactamide monoethanolamine, acetamide monoethanolamine and any combination thereof.

192. The process of claim 189, wherein said obtaining includes heating a mixture of said at least one hydrocarbon alcohol, said at least one fatty alcohol, said at least one surface active agent and said water, at a temperature of at least 30° C.

193. The process of claim 189, wherein said obtaining includes heating a mixture of said at least one hydrocarbon alcohol, said at least one fatty alcohol, said at least one surface active agent and said water, at a temperature of at least 40° C.

194. The process of claim 189, wherein said at least one hydrocarbon alcohol comprises at least one aliphatic alcohol having from 1 to 6 carbon atoms.

195. The process of claim 194, wherein said at least one aliphatic alcohol is selected from the group consisting of methanol, ethanol, n-propanol, isopropanol, n-butanol, sec-butanol, isobutanol and t-butanol and mixtures thereof.

196. The process of claim 189, wherein said at least one fatty alcohol has between 10 and 22 carbon atoms.

197. The process of claim 196, wherein said at least one fatty alcohol is selected from the group consisting of cetyl alcohol, stearyl alcohol, lauryl alcohol, myristyl alcohol, palmityl alcohol and mixtures thereof.

198. The process of claim 189, wherein said at least one surface-active agent is selected from the group consisting of polysorbate 60, ethoxylated sorbitan stearate, ethoxylated sorbitan palmitate, ethoxylated sorbitan oleate, nonyl phenol ethoxylates, fatty alcohol ethoxylates and mixtures thereof.

199. The process of claim 189, wherein said non-CFC propellant is selected from a group of propellants consisting of nitrous oxide, carbon dioxide, nitrogen, propane, iso-butane, n-butane, isopentane, n-pentane, dimethyl ether and mixtures thereof.

200. The process of claim 189, wherein said obtaining comprises:
mixing said water, said at least one fatty alcohol and said at least one surface active agent, so as to obtain a clear aqueous solution; and
adding said at least one hydrocarbon alcohol to said aqueous solution, to thereby obtain said carrier.

201. The process of claim 200, wherein said mixing further includes heating said aqueous solution at a temperature of at least about 40° C.

202. The process of claim 200, wherein said mixing further includes heating said aqueous solution at a temperature of at least about 60° C.

203. The process of claim 200, wherein said adding is performed while heating said aqueous solution at a temperature of at least about 30° C.

204. The process of claim 200, wherein said adding is performed while heating said aqueous solution at a temperature of at least about 39° C.

205. The process of claim 189, wherein said obtaining comprises:
mixing said hydrocarbon alcohol, said at least one fatty alcohol and said at least one surface active agent, so as to obtain a clear alcoholic solution; and
adding said water to said alcoholic solution, to thereby obtain said carrier.

206. The process of claim 205, wherein said adding is performed while heating said alcoholic solution at a temperature of at least about 30° C.

207. The process of claim 205, wherein said adding is performed while heating said alcoholic solution at a temperature of at least about 40° C.
